(19) United States
(12) Patent Application Publication
(10) Pub. No.: US 2011/0053201 A1
(43) Pub. Date: Mar. 3, 2011

(54) METHOD OF CELL SEPARATION

(76) Inventors: Klaus-Gunter Eberle, Tuttlingen (DE); Roland Biset, Leuven (BE)

(21) Appl. No.: 12/602,965

(22) PCT Filed: Jun. 4, 2008
(86) PCT No.: PCT/EP08/56925

§ 371 Date: Mar. 1, 2010

(30) Foreign Application Priority Data
Jun. 5, 2007 (DE) 10 2007 000 309.0

(51) Int. Cl. C12Q 1/02; C12N 5/078; C12M 1/00

(52) U.S. Cl. 435/29; 435/325; 435/308.1

(57) ABSTRACT

A method for the cell separation from blood or from blood products, having the following steps:
(i) inserting a blood bag (35) containing a blood product into a blood bag section (5) of a cartridge (1)
(ii) inserting a tube (36) connected to the blood bag (35) into a product transport path (36) which connects the blood bag (35) to a product bag (33) wherein a first sensor (25) and a second sensor (85) are positioned along the product transport path (36),
(iii) inserting the cartridge (1) into a rotor of a centrifuge,
(iv) spinning the centrifuge for separating the individual blood components,
(v) transporting a predetermined quantity of a blood component along the product transport path (36) at a first flow speed,
(vi) detecting the composition of the transported blood component by the first sensor (25). The second sensor (85) also detects the composition of the transported blood component after the first sensor (25) has output a signal corresponding to a predetermined composition of the blood component. The transporting and the spinning are terminated when the second sensor (85) detects the predetermined composition and outputs a respective end signal, or when a predetermined period of time has elapsed.

### TECHNICAL FIELD

The invention relates to a method for cell separation from whole blood or from a blood product, a cartridge suitable for performing said method and a respective centrifuge.

### STATE OF THE ART

In transfusion medicine, the so-called blood component therapy has established itself since the beginning of the nineties. This means that, instead of a whole blood conserve, only those blood components are administered to the patient that the individual patient requires. By means of this separate administering of the individual blood components it is possible that one single blood conserve can provide optimal help to an average of 1.8 patients.

The essential blood components are
the red blood cells in the so-called erythrocyte concentrate which are transfused in order to maintain the oxygen supply after severe loss of blood.
the blood platelets in the thrombocyte concentrate which are administered in cases of coagulation disturbances (haemophilia), and
the blood plasma which is administered in cases of coagulation disturbances and volume deficits. Apart therefrom, blood plasma is an essential basic component for the production of many medicaments.

The separation of the individual blood components which is defined as cell separation/isolation is known to be effected by treating the blood in a centrifuge. By means of centrifuging the individual blood components are separated and can then be filled separately into the respective containers for further use.

Such centrifuge is for example known from document EP 1 351 772 B1. According to this state of the art, a plurality of cartridges are arranged around a hub in a rotor of a centrifuge. The cartridges are firmly held in the rotor such that the blood bags are centrifuged in an upright position. Inside, the cartridges comprise accommodating devices for accommodating a blood bag containing whole blood and product bags in which the plasma and the erythrocyte concentrate are collected, respectively. In order to avoid a continued flowing and renewed mixing of the products after the individual components have been separated, various clamping means are provided in the cartridge for clamping the individual tubes. Before removing the bags from the cartridge after separation has been effected the individual connecting tubes of the bags must be sealed by appropriate means. Only then can the clamps of the cartridge be opened, the bags be removed and the cartridge get prepared for accommodating a new set of bags.

After the separation and the drawing off of the plasma or the red blood cells, a mixture called “buffy coat” remains in the blood bags. This “buffy coat” consists mainly of platelets as well as white and red blood cells. For obtaining/separating the platelets from this “buffy coat” the latter is diluted with an additive solution and this diluted “buffy coat” is then again separated into its components by centrifuging.

From document WO 03/089027 a system and a method for this purpose are already known. This document discloses a centrifuge in which a ring-shaped bag containing a mixture of “buffy coat” and additive solution is inserted into its single chamber. The blood components are then separated by means of a centrifuging operation and the separated components are transported via a tube line through a filter provided in the area of the hub to a collection container also provided in the area of the hub.

### DISCLOSURE OF THE INVENTION

### Technical Object

It is the object of the invention to provide an improved method for the cell separation from blood or from a blood product that enables a better yield in cell separation and a more economic cell separation.

### Technical Solution

The object of the invention is achieved by means of a method for cell separation according to claim 1, a cartridge according to claim 11, and a centrifuge according to claim 18. Advantageous embodiments of the invention are achieved according to the dependent claims.

In this method for cell separation from blood or from blood products according to the invention, a blood bag containing a blood product is inserted into a blood bag section of a cartridge. Then a tube connected to the blood bag is inserted into a product transport path. The product transport path connects the blood bag to a product bag. A first and a second sensor are positioned along the product transport path. Next, the cartridge is inserted into the rotor of a centrifuge.

Then the centrifuge is spun in order to separate the individual blood components. This step is followed by the transporting of a predetermined quantity of a blood component along the product transport path at a first flow speed. Next, the flow speed can be reduced and the blood component can be transported further on. The spinning of the centrifuge also remains active.

The first sensor detects the composition of the blood component during transportation. However, this is preferably effected only after the possible reducing of the first flow speed.

When a predetermined composition of the blood component is reached the first sensor outputs a respective signal. The second sensor is preferably activated only after the first sensor has output the signal. The second sensor then detects the composition of the blood component and outputs an end signal when it also detects the predetermined composition. Thus, the transportation is terminated. As an alternative to the termination by means of the second sensor, the transportation can also be terminated after a predetermined period of time has elapsed since the first sensor has output the signal. When the transportation is terminated, also the spinning is terminated.

The predetermined composition of the blood component means the blood product to be separated, which includes a predetermined proportion of an unwanted blood product. When the predetermined proportion of the unwanted blood product has been reached in the area of the sensor, this corresponds to the predetermined composition.

Thus, an advantageous positioning of the sensors makes it possible to obtain an optimal quantity of the desired blood product without a proportion of the desired blood product remaining in the tube used for transportation.

Advantageously, the blood component can be transported at a reduced initial speed before transporting the predetermined quantity of the blood component along the product transport path at a first flow speed. This serves to ventilate a filter provided in the product transport path between the first sensor and the second sensor. A reduced initial speed, i.e. a slower flow speed, ensures that air bubbles or other inclusions which might cause the second sensor to output an incorrect signal, do not remain in the filter.

Advantageously, the blood component (product) is led into the filter radially from the outside and from below. Thus, the filtering is effected against the centrifugal force.

Before transporting at the first flow speed and the reduced initial speed is effected, respectively, a tube clamp disposed on the tube can be opened by an operating device provided in the cartridge. The closed tube clamp ensures during the handling of the blood bag that its contents do not enter the tube before cell separation is started.

Advantageously, a tube clamp can be provided alternatively or additionally between the second sensor and the product bag, and can be closed when transporting is terminated. Thus, a termination of the transporting at the optimal moment is secured. The respective tube clamps are then opened by the operating devices when transporting is started and closed by means of the operating devices when transporting is terminated.

The first and second sensors can advantageously be provided as optical sensors. The predetermined composition of the transported blood component can then show a certain proportion of red blood cells, which is detected by the sensors.

For adjusting the flow speed, a pressure acting radially outward can be applied onto the blood bag by means of a pressing element provided at the rotor.

The cartridge and the centrifuge according to the invention are, on the one hand, suitable for the separation of cells and plasma from whole blood, but are also provided for the separation of cells from the “buffy coat” that remains after a known centrifuging operation has been effected.

For this purpose, the “buffy coat” from several blood bags is collected together with an additive solution in a new blood bag and is mixed. The new blood bag corresponds to the blood bag according to the invention. The new blood bag can advantageously be provided with a tube and/or a filter, in particular one provided for the filtering of leukocytes.

### BRIEF DESCRIPTION OF THE ILLUSTRATIONS IN THE DRAWINGS

In the following, one embodiment of the invention is described by means of the Figures showing the following:

FIG. 1, a top view of the cartridge according to the invention,

FIG. 2, a perspective view of the cartridge,

FIG. 3, a perspective view of the cartridge, sectioned along a symmetry line,

FIG. 4, a sectional perspective view of the cartridge, supplementing the view of FIG. 4,

FIG. 5, a further perspective and sectional view of the cartridge,

FIG. 6, a bottom surface of a cover of the cartridge,

FIG. 7, a perspective view of an accommodating box, and

FIGS. 8a to 8c, schematic sectional views of the cartridge, from which the cell separation can be seen, and

FIG. 9 shows a flow of the blood product through the filter.

### WAY(S) TO CARRY OUT THE INVENTION

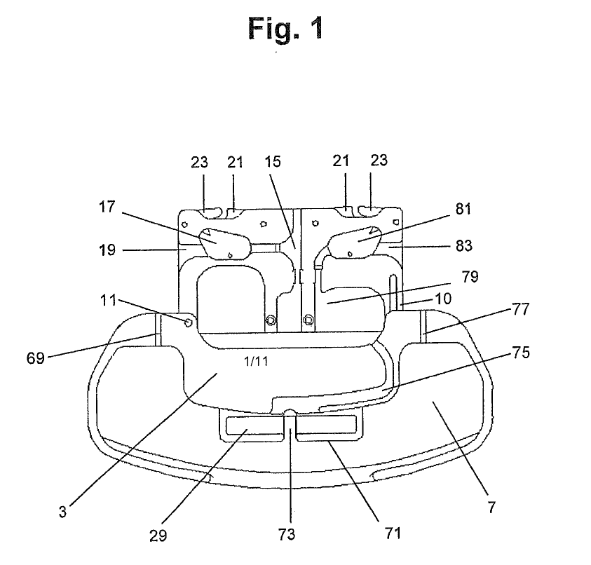

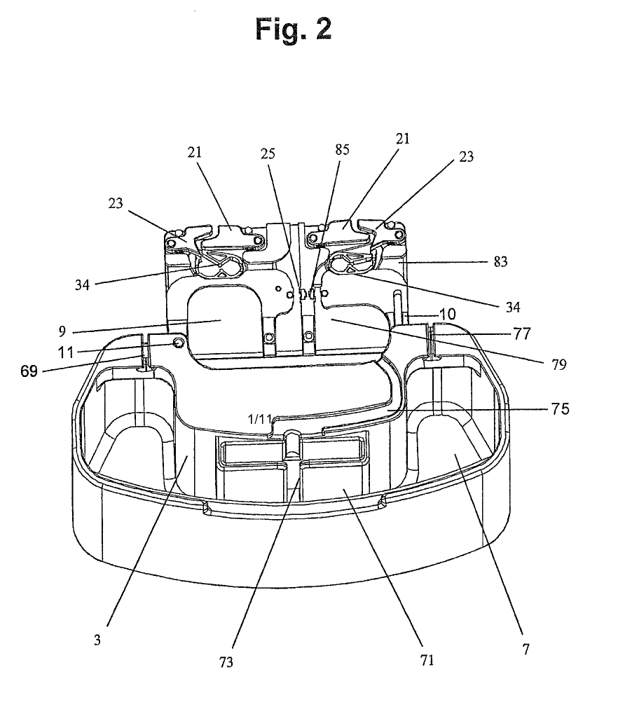

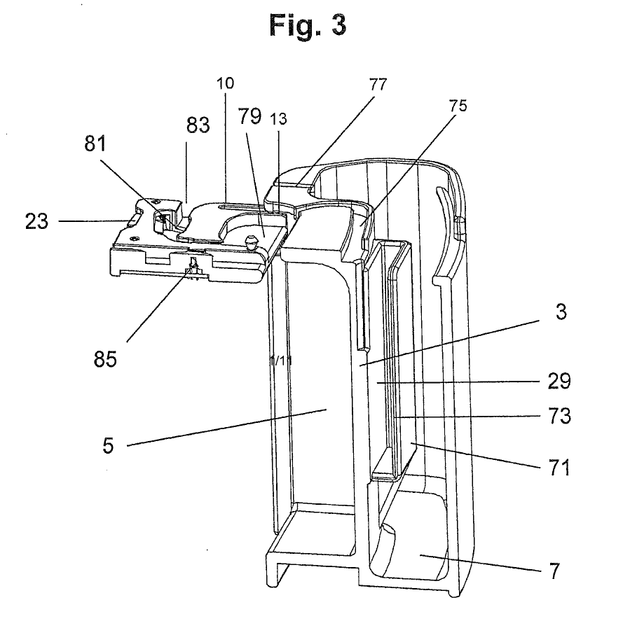

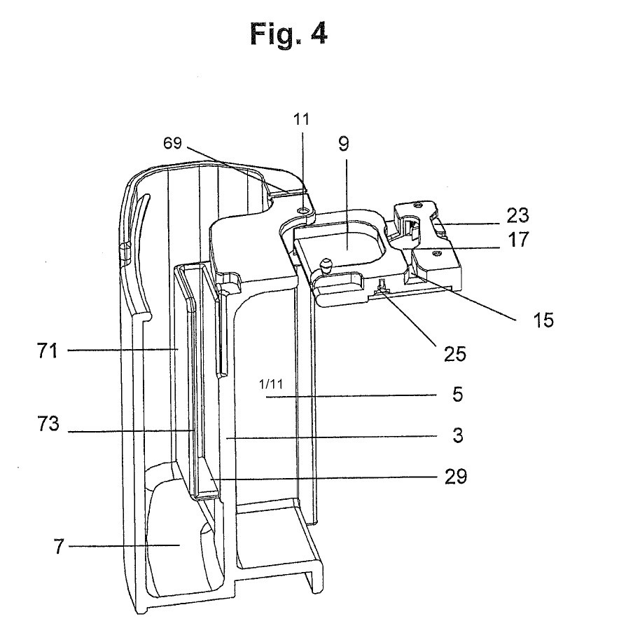

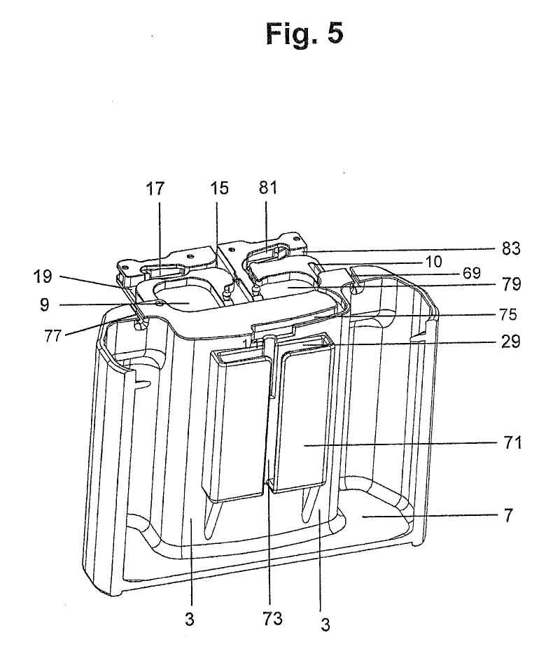

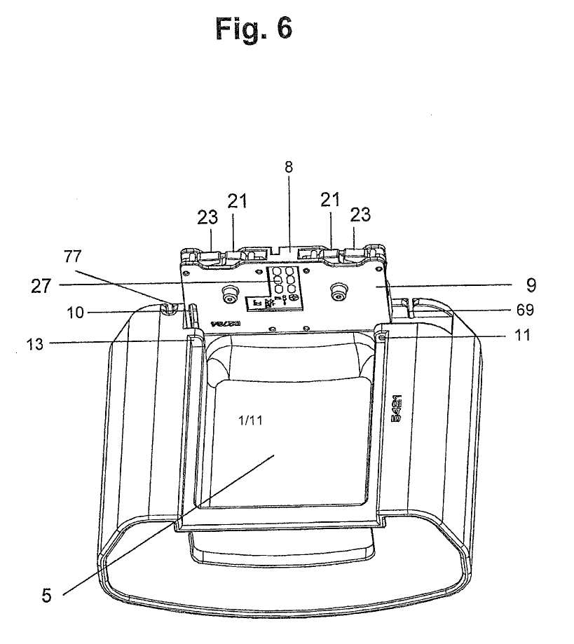

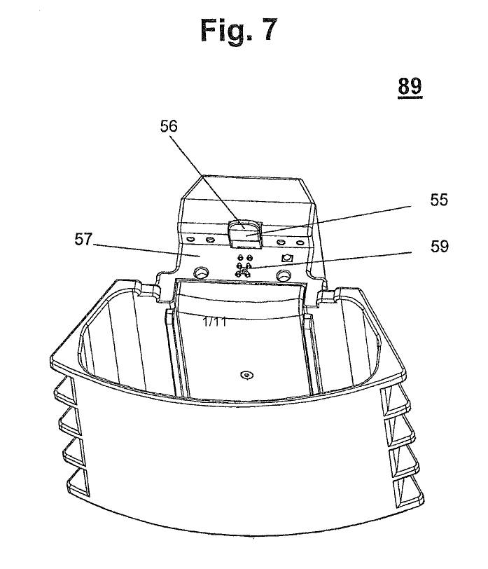

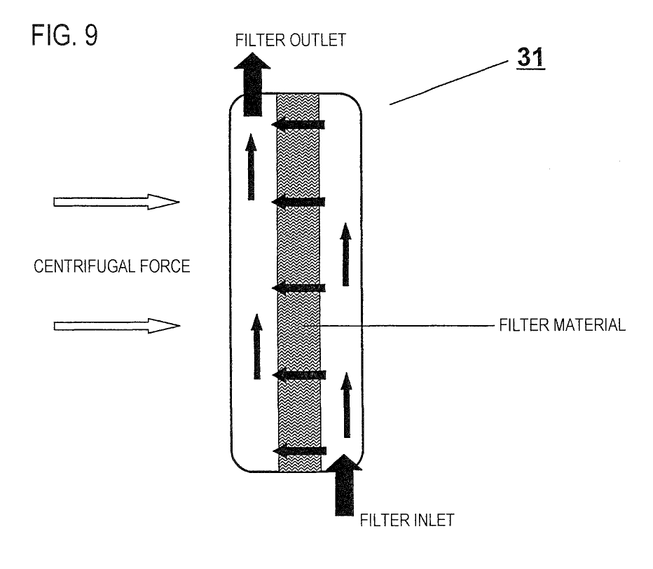

An embodiment of the invention is described by means of FIGS. 1 to 9.

A cartridge 1 essentially consists of a partition wall 3 and a cover 9. The partition wall defines a blood bag section 5 and a product bag section 7. When the cartridge 1 is inserted into a system box 89 of the rotor of a centrifuge, the blood bag section 5 is located radially inside of the partition wall 3, whereas the product bag section 7 is located radially outside of the partition wall 3. An accommodating box 89 according to the invention is designated as system box 89.

A cover 9 is provided above the blood bag section 5. This has an essentially rectangular shape and, in its closed state, one of its longitudinal sides is in contact with the partition wall 3. At one corner point, the cover is pivotally mounted in the partition wall, whereas, at a second corner point, it is engaged with the partition wall 3 by means of a bolt 10. For opening the cover, pressure is applied onto the bolt 10 and then the cover is pivoted to the side. Thus, the blood bag section 5 is freely accessible and can be filled with a blood bag 35.

By means of the simple pivoting mechanism, a tube 36 and a blood bag 35 can quite easily be held in a desired position during when the cover 9 is closed, and can be fixed in this desired position by closing the cover 9.

After the cover 9 has been closed, it is possible to insert the tube 36 into recesses 15, 19 formed in the top surface of the cover 9. A first photo sensor 25 is provided in the recess 15.

A tube clamp 34 in its closed state, which is delivered together with the blood bag and which is disposed on the tube, for example one produced by “Halkey Roberts”, is accommodated in a recess 17 also formed in the top surface of the cover 9.

The end of the tube 36 which is the far end with respect to the blood bag 35 leads to the product bag section 7 and is there connected to a leukocyte filter 31 which is held in a fixture 29. The tube 36 is inserted into the leukocyte filter 31 radially from the outside and from below. The insertion of the filter 31 and the tube 36 is enabled by means of a slot 73 in an outside wall 71 of the fixture 29. Through the slot 73, the tube 36 connected to the filter 31 can be displaced from top to bottom, when the filter is inserted into the fixture, such that the tube leads to the filter radially from the outside and from below.

Behind the filter 31, the tube 36 leads via second recesses 75, 79, 81, 83 which are provided in the cover 9 and which are positioned in an essentially mirror-image manner with respect to the recesses 15, 17, 19 to the product bag 33, which is located radially outside of the fixture 29.

A second tube clamp 34 is provided in the recess 81. A second photo sensor 85 is located in the recess 79.

Inside the cover 9, two rods 21, 23 as operating devices for operating the clamps 34 are respectively led through the cover such that one of their ends slightly protrudes from a side surface 8 of the cover 9, which is located opposite the partition wall 3, and the other end is located in the area of the recess 17 accommodating the clamp 34. By applying a pressure onto one of the ends protruding from the side surface 8, the standard clamp 34 can thus be opened and closed. According to the embodiment, the tube clamps 34 can be operated individually as well as pneumatically.

After the cartridge 1 has been loaded, the cartridge 1 can be inserted into the system box 89 of the rotor of a centrifuge. When this is done, the side surface 8 of the cover 9, which is located opposite the partition wall 3, rests on a support 57 of the system box 89, which is provided in the area of a hub of the centrifuge. At the support 57, there is also a rod-shaped locking element 55 which has a projection 56 at its radial outside. By the insertion of the cartridge 1, the side surface 8 of the cover 9 slides over the projection 56 and moves the locking element 55 radially inward until the side surface 8 is positioned below the projection 56 and the locking element 55 springs back to its original position and thus prevents an upward movement of the cartridge 1. Thus the cartridge 1 is firmly positioned between the outside wall of the system box 89 and the support.

According to the embodiment, the rotor of the centrifuge is designated for six system boxes 89 having one cartridge 1 each. After all cartridges 1 have been inserted, the centrifuge is started. By means of the centrifugal force, the desired separation of the blood components is effected. Since the “buffy coat” diluted by an additive solution is in the blood bag 35, the lighter components of it will remain radially inside, whereas its heavier components, i.e. the red blood cells, collect at the outside.

In order to transport the desired blood component—according to the embodiment, these are the platelets—in high quality, i.e. without the admixture of other blood cells, from the blood bag, the separation of the components will be followed by a slight pressure being applied onto the blood bag by means of a known pressure pad 61, so that, after the clamps 34 have been opened, the solution rich in platelets begins to rise into the tube 36 leading upwards and radially inwards. The solution rich in platelets is led through the tube 36 into the leukocyte filter 31 into which it enters radially from the outside and from below.

In the leukocyte filter 31, the unwanted leukocytes, i.e. the white blood cells, are removed. Due to the arrangement according to the invention of the tube 36 having the filter 31, the filtration is effected against the centrifugal force. Thus, heavier blood components, such as unintentionally transported red blood cells, are trapped in a front-end chamber of the filter, positioned radially outside.

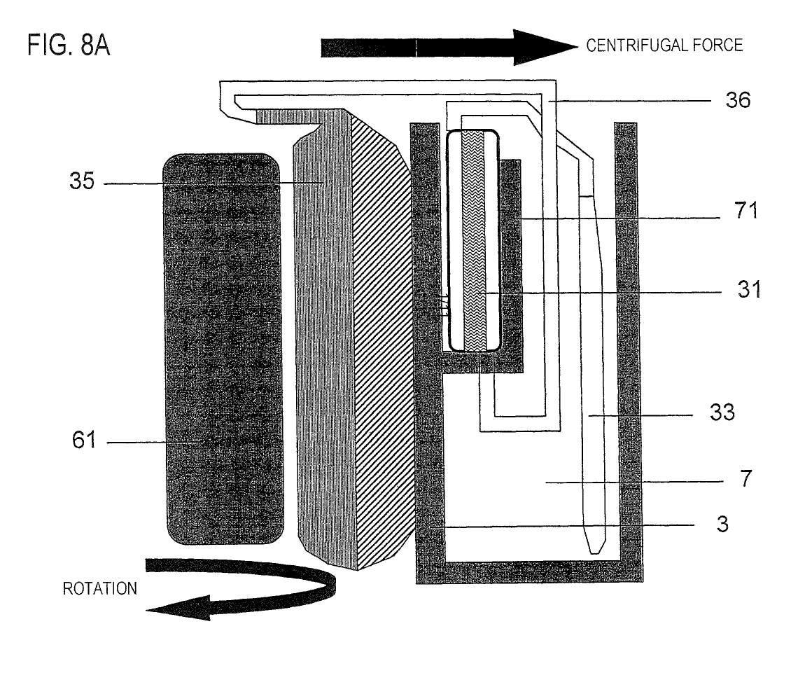

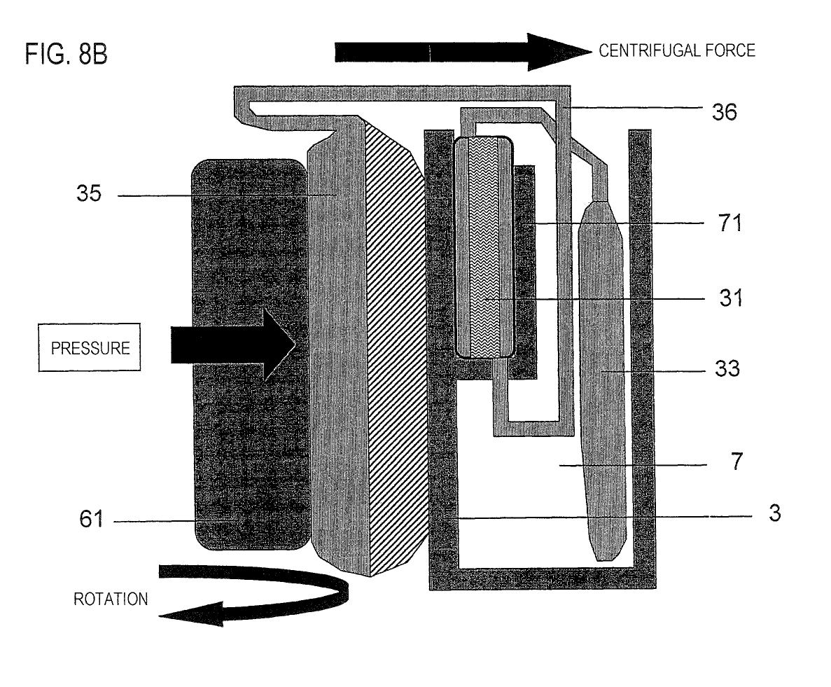

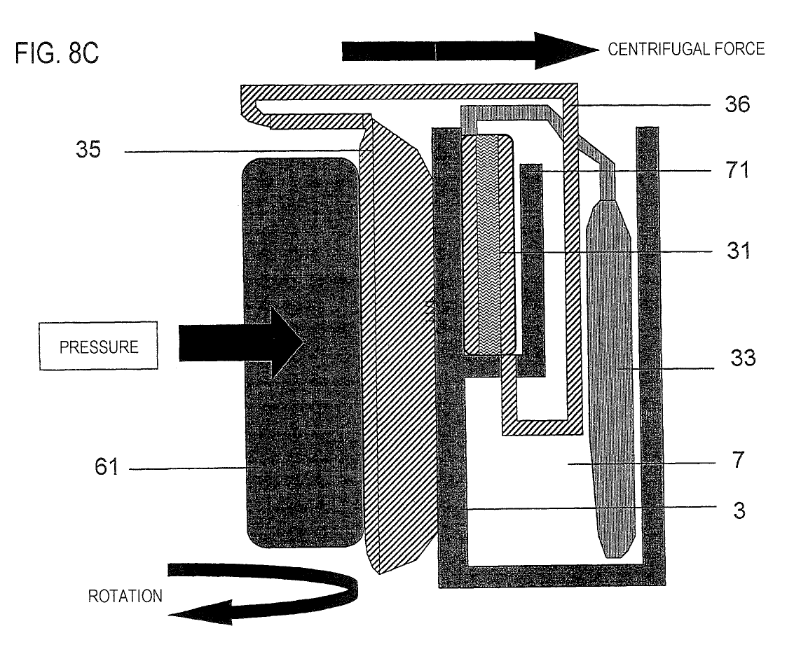

After having passed the leukocyte filter 31, the solution rich in platelets continues flowing through the tube 36 into a product bag 33, in which it is collected. Preferably, the product bag 33 is already formed as final storage bag for the product. The entire process is schematically illustrated in FIGS. 8a to 8c.

In order to remove any air that might be present in the filter, the flow speed is kept low for a certain volume quantity at the beginning of the product transfer, and thus it is enabled that the filter fills reliably and completely with the blood product. After the transfer of this predetermined volume quantity, the transport speed for a second predetermined volume quantity is increased by means of an appropriate control of the pressure pad. While this second volume is transported, there is hardly any risk that red blood cells contaminate the blood product (here: the thrombocyte concentrate). Should this nevertheless happen, this small number of red blood cells is collected in the lower and outer areas of the filter, due to the feeding of the tube from radially outside and below into the filter, and due to the effects of the centrifugal force.

After the second volume has been transferred, the first photo sensor is activated and the flow speed of the blood product in the tube 36 is reduced.

When the first photo sensor 25 detects a predetermined proportion of red blood cells in the thrombocyte-rich solution, it outputs a signal by means of which the flow speed is again reduced. Furthermore, the second photo sensor 85 provided behind the filter 31 is activated.

During this phase, also a rather large number of red blood cells can enter into the filter 31 and even pass through it, until the second photo sensor 85 detects a predetermined proportion of red blood cells in the blood product and outputs a signal for terminating the cell separation process. By means of this signal the tube clamps 34 are closed by activation of the rod 23, so that the red blood cells in the filter are reliably separated from the thrombocyte concentrate in the product bag. The operation of the rod is effected by means of an actuating mechanism provided in the system box 89.

As an alternative to the termination by means of the second photo sensor 85, the cell separation process can also be terminated after a certain period of time has elapsed after the second photo sensor 85 has been activated.

In the embodiment, altogether six cartridges are provided in the centrifuge. The above described control of the cell separation process in a cartridge 1 by means of a pressure pad 61, the opening and closing of the tube clamps 34, and the process control by means of the two photo sensors 25, 85 enables a continued cell separation in the cartridges of the other system boxes 89, since the described process control operates individually for each combination of cartridge and system box.

For the transmission of the control and other electric signals an electric contact pad in the form of a plurality of individual contact points 59 is provided at the support 57 of the system box 89. At the bottom surface of the cover 9, contact areas 27 assigned to the contact points 59 are provided and get into contact with the contact points 59 when the cartridge 1 is inserted into the system box. For this purpose, the contact points 59 are spring-mounted.

For the purpose of an easier handling, on the one hand, and in case blood components should escape due to a damage of the bags 33, 35, the tube 36 or the filter 31, the cartridge 1 is inserted into a collecting tank 87 from a radially inward direction. In case of a damage, the escaping blood component is largely collected in the collecting tank 87 so that there is only little contamination of the system box 89 or of the rotor itself. In such a case, the system box 89 can be easily dismounted from the rotor.

After the cell separation has been terminated, each of the cartridges 1 is removed by applying a slight pressure onto the locking element 55 in order to move this radially to the inside.

Simultaneously, the cartridges 1 are seized at the finger holes 88 of the collecting tank 87 and lifted upwards out of the system box 89 of the centrifuge, and are immediately replaced by new, freshly loaded cartridges 1. During the subsequent cell separation, the blood bags 35 and the product bags 33 can be removed from the exchanged cartridges 1 and these can be reloaded.

A method for the cell separation from blood or from blood products has the following steps:

(i) inserting a blood bag (35) containing a blood product into the blood bag section (5) of a cartridge (1)
(ii) inserting a tube (36) connected to the blood bag (35) into a product transport path (36) which connects the blood bag (35) to a product bag (33), wherein a first sensor (25) and a second sensor (85) are positioned along the product transport path (36),
(iii) inserting the cartridge (1) into a rotor of a centrifuge,
(iv) spinning the centrifuge for separating the individual blood components,
(v) transporting a predetermined quantity of a blood component along the product transport path (36) at a first flow speed,
(vi) detecting the composition of the transported blood component by a first sensor (25). The second sensor (85) also detects the composition of the transported blood component after the first sensor (25) has output a signal corresponding to a predetermined composition of the blood component. The transporting and the spinning are terminated when the second sensor (85) detects the predetermined composition and outputs a respective end signal, or when a predetermined period of time has elapsed.

1. A method for cell separation from blood or from blood products, comprising the following steps:
(i) inserting a blood bag (35) containing a blood product into a blood bag section (5) of a cartridge (1),
(ii) inserting a tube (36) connected to the blood bag (35) into a product transport path (36) which connects the blood bag (35) to a product bag (33), wherein a first sensor (25) and a second sensor (85) are positioned along the product transport path (36),
(iii) inserting the cartridge (1) into a rotor of a centrifuge,
(iv) spinning the centrifuge in order to separate the individual blood components,
(v) transporting a predetermined quantity of a blood component along the product transport path (36) at a first flow speed,
(vi) detecting the composition of the transported blood component by the first sensor (25),
characterized in that,
after the first sensor (25) has output a signal corresponding to a predetermined composition of the blood component, the second sensor (85) also detects the composition of the transported blood component, and the transporting and the spinning are terminated when the second sensor (85) detects the predetermined composition and outputs a corresponding end signal, or when a predetermined period of time has elapsed.

2. A method for cell separation according to claim 1, wherein the blood component is transported at a reduced initial speed before step (v) in order to ventilate a filter (31) provided in the product transport path (36) between the first sensor (25) and the second sensor (85).

3. A method for cell separation according to claim 1, wherein the product is transported to the filter (31) radially from the outside and from below and the filtration is effected against the centrifugal force.

4. A method for cell separation according to claims 1 to 3, wherein a tube clamp (34) disposed on the tube (36) is opened by an operating device (21, 23) provided in the cartridge (1), before transporting at the first flow speed and at the reduced initial speed is started, respectively.

5. A method for cell separation according to claims 1 to 4, wherein a first tube clamp (34) is provided between the first sensor (25) and the filter (31) and/or a second tube clamp (34) is provided between the second sensor (85) and the product bag (33).

6. A method for cell separation according to claim 5, wherein the first tube clamp (34) and/or the second tube clamp (34) are/is opened when transporting is started, and is closed when transporting is terminated, by means of the operating device (21, 23).

7. A method for cell separation according to one of claims 1 to 6, wherein the first and the second sensors (25, 26) are optical sensors, and the predetermined composition of the transported blood component comprises a certain proportion of red blood cells.

8. A method for cell separation according to one of claims 1 to 7, wherein for adjusting the flow speed a pressure acting radially outward is applied onto the blood bag (35) by means of a pressing element (61) provided at the rotor.

9. A method according to one of claims 1 to 8, wherein the second sensor (85) is only activated after the first sensor (25) has output a signal indicating the predetermined composition.

10. A method according to one of claims 1 to 9, wherein the first flow speed is reduced after a predetermined period of time has elapsed or when a predetermined transported volume has been reached, and the blood component is further transported at the reduced flow speed, and the first sensor detects the composition of the blood component after the flow speed has been reduced.

11. A cartridge to be inserted into a centrifuge for the cell separation from blood or from blood products, comprising:
a blood bag section (5) for inserting a blood bag, a product transport path (36) which connects the blood bag (35) to a product bag (33), wherein a first sensor (25) and a second sensor (85) are positioned along the product transport path (36),
characterized in that
the first sensor (25) and the second sensor (85) are provided in series, and the second sensor (85) can be activated to detect the composition of the transported blood component after the first sensor (25) has output a signal indicating a predetermined composition of a blood component transported in the product transport path, and the transporting is terminated when the second sensor (85) detects the predetermined composition and outputs a corresponding end signal, or when a predetermined period of time has elapsed.

12. A cartridge according to claim 1, wherein a filter (31) is provided between the first sensor (25) and the second sensor (85).

13. A cartridge according to claim 1, wherein the product transport path leads to the filter (31) radially from the outside and from below.

14. A cartridge according to claims 1 to 3, wherein a tube (36) on which a tube clamp (34) is disposed is provided in the product transport path, said tube clamp being operable by an operating device (21, 23) provided in the cartridge (1).

15. A cartridge according to claims 1 to 4, wherein a first tube clamp (34) is disposed between the first sensor (25) and the filter (31) and/or a second tube clamp (34) is disposed between the second sensor (85) and the product bag (33).

16. A cartridge according to claim 5, wherein the first tube clamp (34) and/or the second tube clamp (34) are/is opened when transporting is started, and are closed, when transporting is terminated, by the operating device (21, 23).

17. A cartridge according to one of claims 1 to 6, wherein the first and the second sensors (25, 26) are optical sensors.

18. A centrifuge having a cartridge (1) according to one of claims 11 to 17, wherein, for adjusting the flow speed, a pressing element (61) disposed at the rotor is provided for applying a pressure acting radially outward onto the blood bag (35).
